(19) United States
(12) Patent Application Publication
(10) Pub. No.: US 2008/0003670 A1
(43) Pub. Date: Jan. 3, 2008

(54) High density permeable supports for high throughput screening

(75) Inventors: Gregory Roger Martin, Acton, ME (US); Allison Jean Tanner, Portsmouth, NH (US)

Correspondence Address: CORNING INCORPORATED, SP-TI-3-1, CORNING, NY, 14831, US

(73) Assignee: Corning Incorporated

(21) Appl. No.: 11/479,533

(22) Filed: Jun. 30, 2006

(51) Int. Cl. C12M 1/34; C12M 3/00

(52) U.S. Cl. 435/297.5; 435/288.4

(57) ABSTRACT

A class of designs is provided for a permeable support where the design principle includes a permeable support layer, such as a membrane, bonded midway between two arrays of wells or multiwell plates, attached at their bottom portions, forming a double-sided multiwell plate. Thus, opposite facing wells on either side of the permeable support layer are accessible by inversion of the double-sided multiwell plate. Well fluid is held in place in the novel multiwell plate by capillary forces in the case of aligned upper and lower well arrays or by surface tension on patterned well regions on a permeable membrane layer. No additional components are necessary to form compartments for fluid retention. These new plate designs allow the surface area of the permeable support layer to be maximized, eliminate potential wicking and cross contamination issues that may arise from multiple component sidewalls, and take advantage of the small well diameters to retain fluid by utilizing the effects of surface tension or capillary forces.

### FIELD OF THE INVENTION

This invention relates generally to cell culture labware, such as multiwell plates, and more particularly to a class of designs for Transwell® Permeable Supports or permeable supports within multiwell plates useful for high throughput screening.

### BACKGROUND OF THE INVENTION

Multiwell plate systems that include permeable supports (or membranes), such as those found in Corning® Transwell® Permeable Supports, facilitate assessment of new chemical entities through the use of high-throughput screening. These multiwell plate systems with permeable supports are used for many types of studies, including drug transport and cell migration. In drug transport studies, intestinal cells (e.g. CaCO2) are grown into a confluent monolayer on the permeable support until they differentiate to form tight junctions. A molecule of interest is then introduced on one side of the cell sheet to see if it can be actively transported across the sheet to the other side where it is detected.

Permeable supports are also useful for cell migration studies, where a chemo-attractant is placed in a compartment adjoining a cell monolayer, and the migration of cells toward the chemo-attractant is detected. Chemical entities are typically tested to determine the ability to block migration.

Alternatively, permeability can be assessed using techniques known in the art such as Parallel Artificial Membrane Assay (PAMPA) or Immobilized Artificial Membranes (IAM). No cell culture is necessary for these applications. Instead, the permeable support is typically coated with a solution of lipid in an inert organic solvent. The molecule of interest is placed on one side of the coated permeable support and its passage to the other side is monitored.

High throughput screening permeable support systems are currently available with 96 well tests being carried out in parallel, and require multiple components including a Transwell plate and a receiver or reservoir plate in which the Transwell resides. In order to increase throughput, there is interest in the field to increase the well density of the multiwell plate, to higher densities such as 384 well and 1536 well formats, for example. In the current art, the permeable support is typically suspended at the bottom of a well. The well or multiwell plate with the permeable support is then placed inside another well or reservoir plate having a solid bottom, enabling fluid retention. The increase in the number of wells leads to a miniaturization of the wells in these higher density formats; accordingly, the total membrane area for cell growth within the well becomes limited and the close proximity of the sidewalls in the multiple components can lead to wicking or cross contamination issues.

The design and manufacturing challenges seen with 96 wells become even more pronounced and difficult as one proceeds to 384 and 1536 well formats. Hence, it is desirable to overcome the problem of wicking or contamination in multiwell plates. Additionally, it is desirable to reduce the number of discrete parts in the permeable support.

Prior art approaches for performing the above described desired capabilities that are known in the art include the following examples.

Different types of filtration devices are found in U.S. Pat. No. 5,047,215, entitled, “Multi Well Test Plate”; U.S. Pat. No. 4,304,865, entitled “Harvesting Material from Micro-Culture Plates”; and U.S. Pat. No. 4,948,442, entitled “Method of Making a Multi Well Test Plate”.

The above prior art patents disclose filtration devices which have some disadvantages. These prior art patents disclose methods of making microtiter filtration plates by sealing a sheet of filter material between thermoplastic trays or require cutting the filter material from the microtiter plates into discrete discs for analysis. If the filter material is left as an uncut sheet, wicking and cross-contamination occur because the filter material has a pore structure that runs laterally.

Accordingly, first off, these prior art devices have great difficulty solving the problem of cross contamination and wicking. Secondly, because of the problem arising if the sheet is uncut, these prior art designs necessitate the use of many discrete components. Thirdly, in so doing, they are not easily fabricated. Fourthly, they do not provide for the maximum surface area of a permeable support or for miniaturization to allow for increased well density formats.

Another piece of prior art entitled, “Reversible Membrane Insert for Growing Tissue Cultures”, U.S. Pat. No. 5,759,851, assigned to the assignee hereof, discloses a well with a membrane inside and at the bottom of it, where the well is then placed in a separate reservoir well. In this type of device, two separate containers are necessary: a membrane-containing well and a reservoir well, much like Transwells which are known in the art. Cells can be seeded in the membrane-containing well on the membrane's upward facing side where, as mentioned above, the membrane is located at the bottom of the well. After these cells become adherent, the membrane-containing well can be removed from the reservoir, and using a jig tool, the membrane itself can then be moved to the opposite end of the membrane-containing well. The membrane-containing well is placed inverted into the reservoir thereby allowing cells to seed on the other side of the membrane, the side without cells already seeded. Accordingly, this enables seeding cells on both sides of the membrane.

One of the major disadvantages with this design is that separate, detached wells or containers are needed, one membrane-containing well and one reservoir well. Furthermore, an additional device, such as a jig, is required to move the membrane from one end of the well to the other prior to inverting and seeding with cells.

Accordingly, a new apparatus and method of manufacture is needed that preferably overcomes the disadvantages of any of the prior art solutions above-mentioned that provides a resolution to the problem of cross-contamination and wicking, maximizes the surface area of the permeable supports, allows for miniaturization to increased well density formats, reduces the number of necessary components and simplifies manufacturing/fabrication while also enabling the seeding of cells on both sides of the membrane if necessary.

### SUMMARY OF THE INVENTION

A class of designs is provided for a double-sided multiwell plate where the design principle includes opposite facing wells on either side of a permeable support layer or membrane, where each side is accessible by inversion of the double-sided multiwell plate. Well fluid is held in place by capillary forces in the case of aligned upper and lower array wells or multiwell plates or by surface tension on patterned well regions of a permeable membrane.

One embodiment of the present invention relates to a plurality of first wells forming a first array, a plurality of second wells aligned with the first array of first wells, forming a second array, and bottom portions of the first and second arrays of wells coupled together and having a permeable membrane at their interface.

Additionally, each well of the first array has a respective well opening at the top of each well, these respective well openings facing up; and each well of the second array has a respective well opening at the top of each well, these respective well openings facing down.

Another embodiment of the present invention relates to the well openings of the second array being accessible by flipping the multiwell plate upside-down, thereby positioning the second array such that the respective well openings at the top of each well are facing up and positioning the first array such that the respective well openings at the top of each well are facing down. Fluid in the wells of both the first and second arrays is retained within the wells due to surface tension or capillary forces.

One embodiment of the present invention relates to bonding of the permeable membrane midway through the first and second arrays of wells and wherein the permeable membrane is preferably a track-etch membrane.

Another aspect of the embodiment of the present invention relates to a rigid lid for covering the well openings of the multiwell plate, where the lid is compatible for robotic handling. Yet another aspect is that the lid is a gas permeable lid on at least a portion of the lid and including thin polymer sheets or discs that allow for gas exchange. The lid further includes an elastomeric gasket that fits into a recess on the multiwell plate.

Another aspect of the present invention relates to a sleeve-type lid covering the well openings of the first and second arrays of the multiwell plate, where the sleeve-type lid is thermoformed or molded from a polymer and includes means for accessing the multiwell plate with gripper cut-out areas on top and bottom sides of the sleeve-type lid. In yet another aspect of the present invention, the sleeve-type lid is rigid and gas-permeable on at least a portion of the lid.

Another embodiment of the present invention relates to wells of first and second arrays being patterned onto the permeable membrane to enable cell binding wherein the patterning may be a hydrophilic interaction, specific molecular interaction, coating with a lipid solution, or lamination, onto the permeable membrane.

A still further aspect of the present invention relates to the permeable membrane including non-well regions patterned around the first and second well arrays onto the permeable membrane to prevent cell binding or coating with lipid solutions wherein the patterning may be a coating or lamination with a hydrophobic material. Another aspect of the present invention relates to the patterned permeable membrane being substantially flat.

Additional features and advantages of the invention will be set forth in the detailed description which follows, and in part will be readily apparent to those skilled in the art from the description or recognized by practicing the invention as described in the written description and claims hereof, as well as the appended drawings.

It is to be understood that both the foregoing general description and the following detailed description are merely exemplary of the invention, and are intended to provide an overview or framework to understanding the nature and character of the invention as it is claimed.

The accompanying drawings are included to provide a further understanding of the invention, and are incorporated in and constitute a part of this specification. The drawings illustrate one or more embodiment(s) of the invention, and together with the description serve to explain the principles and operation of the invention.

### BRIEF DESCRIPTION OF THE DRAWINGS

The invention is further illustrated with reference to the following drawings in which:

FIG. 1 is a perspective view of a multiwell plate format in accordance with a preferred embodiment of the present invention.

FIGS. 2 and 3 are cross-sectional views of FIG. 1.

FIG. 4 is a cross-sectional view of one well of FIG. 2 showing the separation by a permeable membrane in accordance with a preferred embodiment of the present invention.

FIG. 5 is a multiwell plate in accordance with an alternate preferred embodiment of the present invention.

FIG. 6 is a cross-sectional view of one well region of FIG. 5.

FIG. 7 is a multiwell plate lid in accordance with a preferred embodiment of the present invention.

FIG. 8 is the underside view of the multiwell plate lid shown in FIG. 7.

FIG. 9 is a gas permeable multiwell plate lid in accordance with a preferred embodiment of the present invention.

FIG. 10 is the underside view of the multiwell plate lid shown in FIG. 9.

FIGS. 11 and 12 show a sleeve type multiwell plate lid covering both upper and lower well openings in accordance with a preferred embodiment of the present invention.

### DETAILED DESCRIPTION OF THE PREFERRED EMBODIMENTS

Reference will now be made in detail to the present preferred embodiments of the invention, examples of which are illustrated in the accompanying drawings. Wherever possible, the same reference numbers will be used throughout the drawings to refer to the same or like parts.

The terms “well” and “well region” as used throughout are intended to signify areas in the novel multiwell plates herein described in which seeding of cells is enabled. Accordingly, these terms are thought to be equivalent and may be used interchangeably throughout.

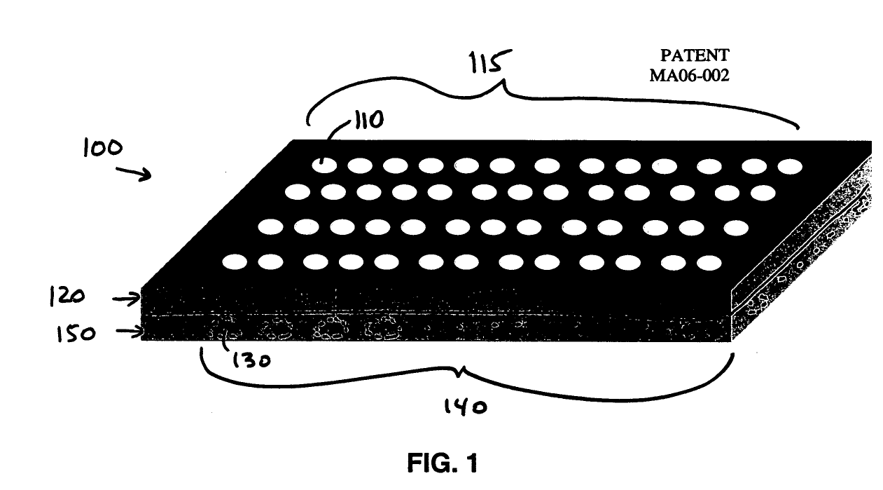

Referring to FIG. 1, a novel double-sided multiwell plate 100 is shown in accordance with a preferred embodiment of the present invention. The standard format of multiwell plate 100 is preferably a high density well format, such as a 384 or 1536 well plate, to increase throughput. The double-sided multi-well plate 100 is preferably one plate that can be utilized on both sides. It is preferably formed from arrays of upper and lowers wells, which may be one plate having upper and lower well arrays or two multiwell plates assembled together into one plate and in either case with a permeable support layer disposed in between (as will be discussed below). For clarity and to distinguish between the upper and lower sides of the double-sided multiwell plate 100, the terms upper and lower multiwell plates will be used herein. Accordingly, multiwell plate wells 110 form an array of wells 115 on an upper multiwell plate 120. Additionally, multiwell plate 100 includes wells 130 found on a lower multiwell plate 150 which form an array 140 (shown with dashed circles) aligned with the wells on the upper multiwell plate. For simplicity, a fewer total number of wells 110, 115, 130, 140 are shown on upper and lower multiwell plates 120 and 150 than are actually physically possible in a standard format of a multiwell plate 100 (e.g. 384 well plate).

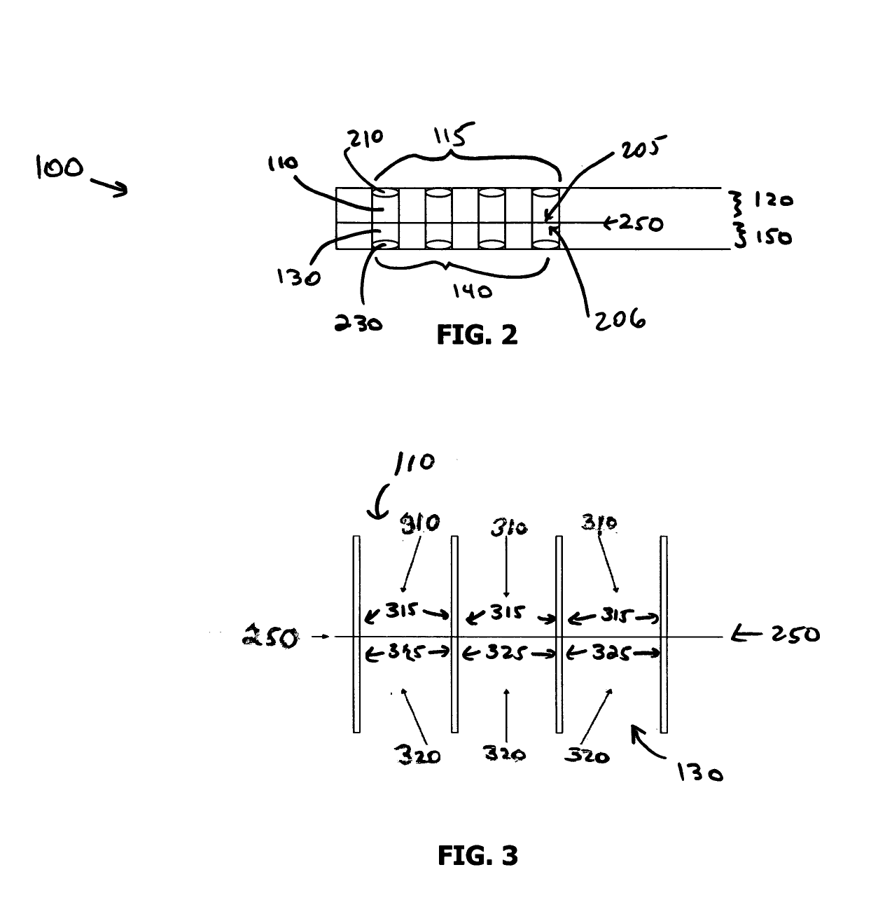

Referring now to FIG. 2, a cross-sectional side-view segment of FIG. 1, multiwell plate 100 is shown to include an upper multiwell plate 120 and lower multiwell plate 150, each with an array of wells 115 and 140, respectively. The bottom portions 205, 206 of the wells of the upper and lower multiwell plate arrays are joined such that the bottom portion of each of the upper wells 205 is aligned with the bottom portion of each of the lower wells 206. Accordingly, the openings 210 of the wells 110 in the upper array 115 are facing up and the well openings 230 of the wells 130 in the lower array 140 are facing down. The well openings 230 of the lower array are accessible by flipping the multiwell plate 100 upside-down, thereby positioning the lower array such that the top of each well 230 in the lower array 140 is now facing up and positioning the upper array 115 such that the top of each well 210 in the upper array is now facing down.

In accordance with a preferred embodiment of the present invention, a permeable support 250 layer, preferably a permeable or porous membrane such as a track-etch membrane, (for example, consistent with supports found in Corning® Transwells® Permeable Supports), is attached or bonded, preferably midway between upper and lower multiwell plates 120 and 150 or at the interface of the bottom portions 205, 206 of upper and lower well arrays. The permeable support layer or membrane 250 may also be treated or coated. Coating materials may include biological materials such as a collagen or various lipids in organic solvents (for PAMPA or IAM applications). Track-etch membranes may be a desirable design choice for those of skill in the art because their size, shape, and density of pores may present advantageous characteristics over other membrane materials for cell transport and migration assays. The pore size of the permeable support layer 250 is typically 1 micron pore size, but can range from 0.1 to 12 microns depending on its end use. The permeable support layer 250 or membrane is stationary, and as such, no extra device is required to move the membrane. Further, the instant invention allows cell seeding to occur on both sides of the permeable membrane 250, with the mere inversion of multiwell plate 100.

Upper and lower multiwell plates 120 and 150 may be joined together via molded fittings as part of the mold design or via known assembly methods such as the use of adhesive, overmolding, solvent bonding, and welding (ultrasonic, RF, laser, platen, radiation, etc.). When joined together, the thickness or height of the multiwell plate 100 may be slightly taller than the traditional multiwell plate, but preferably falls within the range of 14 to 22 millimeters.

Referring to FIG. 3, another cross-section of multiwell plate 100 is shown where the upper and lower multiwell plates 120 and 150, respectively, are shown as being capable of retaining fluid within the wells 110 and 130 due to surface tension or capillary forces. The upper well array 115 has upper fluid compartments 310 within each upper well 110 and the lower well array 140 has lower fluid compartments 320 within each lower well. The compartments 310 and 320 are divided by the permeable support layer 250. Communication between fluids retained in upper and lower fluid compartments 310 and 320 occur through the permeable support 250. The upper fluid compartments have walls 315 and the lower fluid compartments have walls 325. The compartment walls 315 and 325 may also be treated, coated, or textured in such a way to facilitate retention of fluid in the areas immediately adjacent to the permeable support or membrane 250.

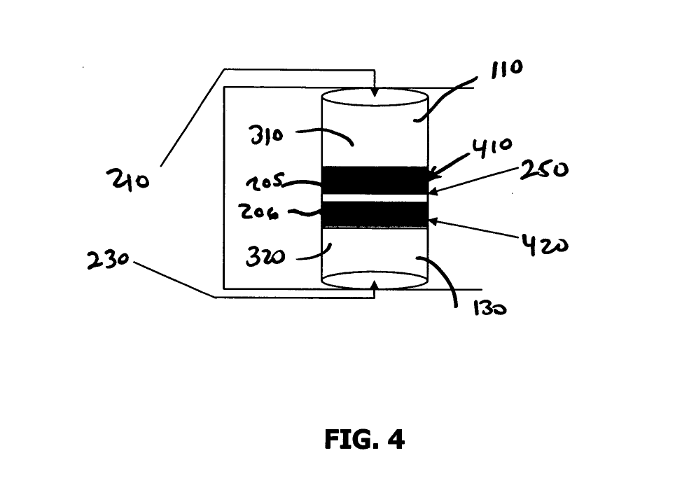

Referring now to FIG. 4, a close up view of one upper well 110 and one lower well 130 is shown. The multiwell plate wells 110, 130 are divided in half by a permeable support layer 250 or membrane as discussed above. The permeable support layer 250 is attached or bonded at the interface of the bottom portions 205, 206 of the upper well 110 and lower well 130, respectively. Upper fluid 410 in upper fluid compartment 310 and lower fluid 420 in lower fluid compartment 320 stay within wells 110 and 130, respectively, due to capillary forces. No additional reservoir is needed in the instant invention, as in the prior art, since upper and lower fluids 410 and 420 will be held next to the membrane 250 on both sides. As discussed above, well openings 210 and 230 can be accessed by flipping the multiwell plate 100 upside-down or inverting it to the opposite side.

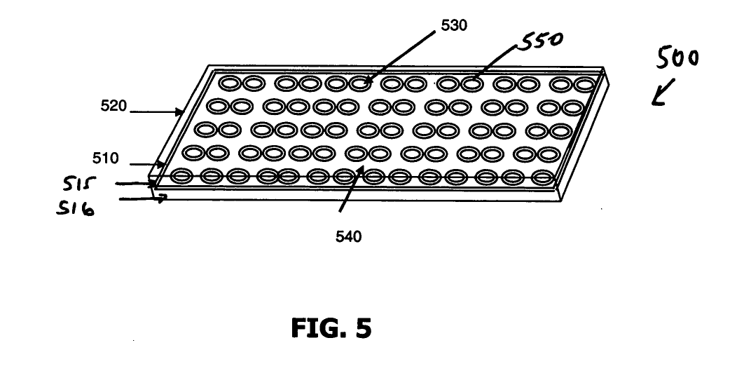

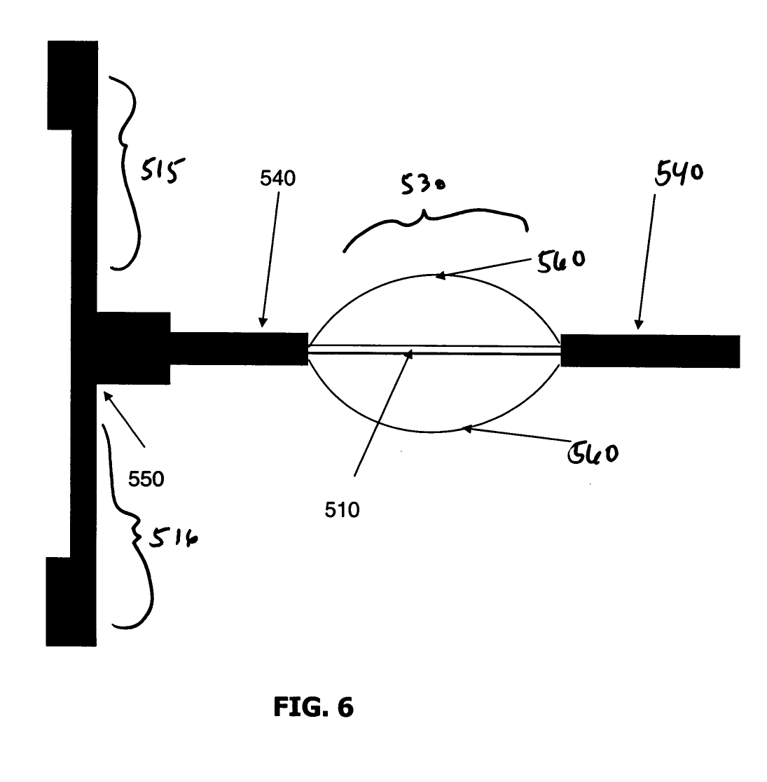

As miniaturization of multiwell plate wells increases in the industry, in particular to provide higher density formats, the total membrane area for cell growth is decreased since the wells may become too small in size to be of utility and further as mentioned above, the close proximity of the sidewalls in the multiple components can lead to wicking or cross-contamination problems. Accordingly, an alternate preferred embodiment of the instant invention provides for a permeable membrane layer or sheet, patterned to retain fluids, attached in a rigid frame in an alternate multiwell plate 500, as shown in FIGS. 5 and 6.

Following the same principles as described supra in previous embodiments, a permeable or porous membrane 510 is held at, preferably, the midpoint of a rigid frame 520 dividing the plate 500 into upper and lower multiwell “plates” (or sides or arrays), 515, and 516, respectively. The membrane 510 is preferably a track-etch membrane. The two sides of the permeable membrane 510 accommodate both upper and lower multiwell plates, 515 and 516 respectively. The permeable membrane 510 is modified or patterned such that a pattern of well regions (or wells) 530 are created that permit cell binding for drug transport and cell migration assays and non-well regions 540 (or non-wells) that prevent cell binding on both upper (shown) and lower sides (not shown) of the permeable membrane 510; non-well regions 540 are disposed around well regions 530.

Preferably, the modification or patterning of the permeable membrane is accomplished with an array of hydrophilic well regions 530 with a hydrophobic grid surrounding them. For PAMPA and/or IAM applications, this can be achieved by a simple hydrophobic membrane spotted with a lipid or organic solvent. The lipid produces hydrophilic spots or well regions. In cell-based testing, where lipids are not spotted or printed, a hydrophilic membrane may be used initially with a hydrophobic material, such as a punched sheet of polyethylene or polypropylene, laminated to the hydrophilic membrane material to create the well regions 530 and non-well regions 540, respectively.

Accordingly, the patterning of the membrane 510 may be completed by a hydrophobic or hydrophilic interaction, a specific molecular interaction, coating or spotting with a lipid or organic solvent, or lamination on top of the permeable membrane or in any other plausible manner.

The well regions 530 that permit cell binding and/or are coated with lipid solution are preferably aligned such that they are located in the same place on either side of the permeable membrane 510, and are preferably in the same location as the wells found on any format of an industry standard multiwell plate for automation compatibility purposes, though the number of wells per plate can vary widely and with any spacing desired.

As mentioned, the non-well regions 540 of the permeable membrane 510 may be modified or patterned by coating or lamination of another material to the permeable membrane, to prevent cell binding and/or coating with lipid solutions. This modification to prevent cell binding and/or coating with lipid solution may also generate demarcations of additional material 550 outlining the perimeter of well regions 530, particularly if lamination is the method of making these well regions, so that the delineation between regions 530 and 540 of the permeable membrane 510 are readily apparent.

One further aspect of the alternate preferred embodiment of the present invention is that despite being modified or patterned, the permeable membrane 510 is substantially flat.

In FIG. 6, a cross-section of the alternate multiwell plate 500 of FIG. 5 is shown depicting a single well region 530 located between non-well regions 540 where a porous or permeable membrane 510 is shown disposed at the midpoint of the multiwell plate 500, held by a rigid frame 550. As mentioned, regions 530 and 540 are patterned to enable or prevent cell binding, respectively, and/or coated with lipid solutions in accordance with preferred aspects of the present invention.

As with embodiments described above, fluid 560 on both sides of the permeable membrane 510 is held in place by surface tension. The rigid frame 550 of alternative multiwell plate 500 preferably has the size and footprint of an industry standard multiwell plate and as with multiwell plate 100, can be inverted for accessing the well regions of the permeable membrane 510 in the lower array 516.

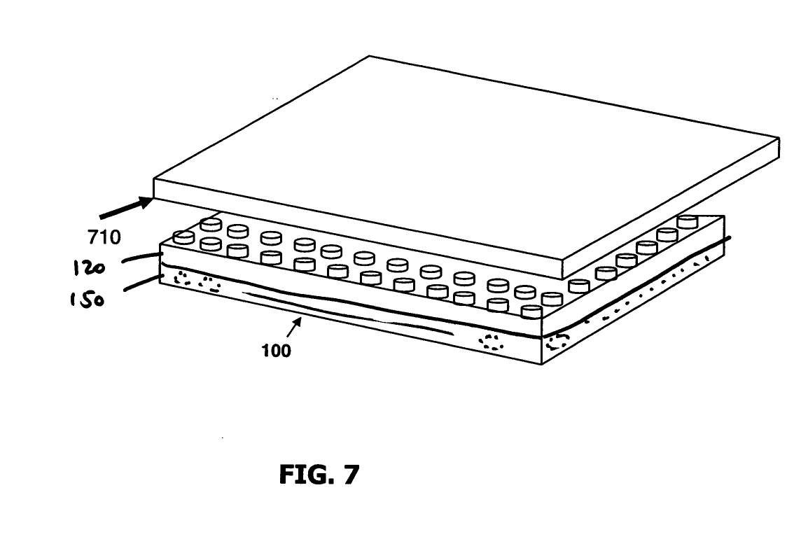

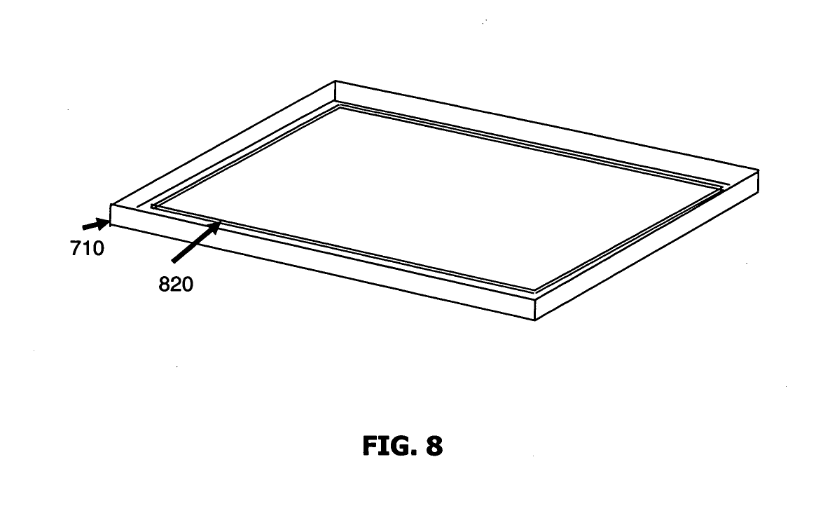

In accordance with preferred embodiments of the present invention, FIGS. 7-12 show different types of lids for covering the upper and lower arrays of multiwell plate 100 (and/or alternate multiwell plate 500) and are capable of fitting a multiwell plate format with any number of wells, though for demonstration and clarity purposes, fewer wells than an industry standard plate are shown Additionally, since multiwell plate 100 and alternate multiwell plate 500 of the instant invention are “open” on both sides, these lids are contemplated to be used to cover both sides of the multiwell plates disclosed herein.

FIG. 7 shows a lid 710 covering the upper array of multiwell plate 100 or the multiwell plate 500 of FIG. 5. Lid 710 is preferably a common rigid multiwell plate lid compatible for robotic handling. In order to prevent evaporation, preferably two such lids 710 will be needed in the instant invention, one for covering each side of either the double-sided multiwell plate 100 or 500. FIG. 8 shows the inverted (or underside view) of lid 710. Also shown in accordance with a preferred aspect of the present invention is an elastomeric gasket 820 on the inside or around the inside perimeter of lid 710, allowing the lid 710 to fit into a recess on the multiwell plate 100. The gasket 820 is preferably made of a thermoplastic elastomer material.

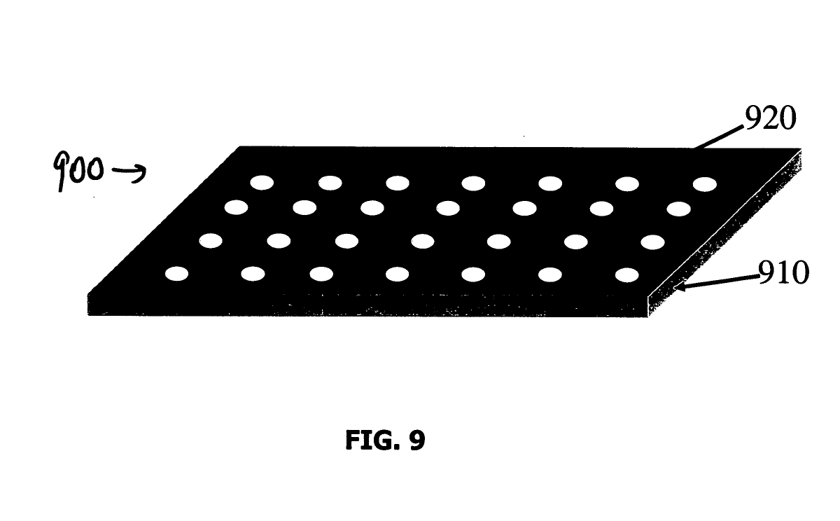

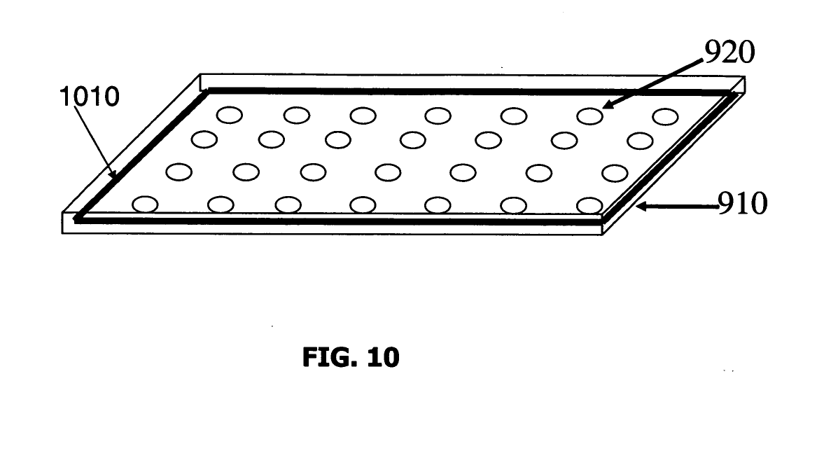

In an alternate preferred embodiment of the present invention, a gas permeable multiwell plate lid 900 for cell based assays is shown in FIG. 9 having a rigid lid portion 910 with holes overlying the wells or well regions and discs 920 made of gas permeable material covering those wells or well regions and allowing for gas exchange. A gas exchange environment may also be provided by a thin polymer sheet (not shown) across the surface of the lid 900 in lieu of discs 920. FIG. 10 shows an inverted view of lid 900 in FIG. 9, where the inverted rigid lid 910 has discs 920 and also shows an elastomeric gasket 1010 around the inside perimeter of lid 910, allowing the lid 910 to fit into a recess on the multiwell plate 100 (or alternate multiwell plate 500). Gasket 1010 is similar in construction to gasket 820 of FIG. 8.

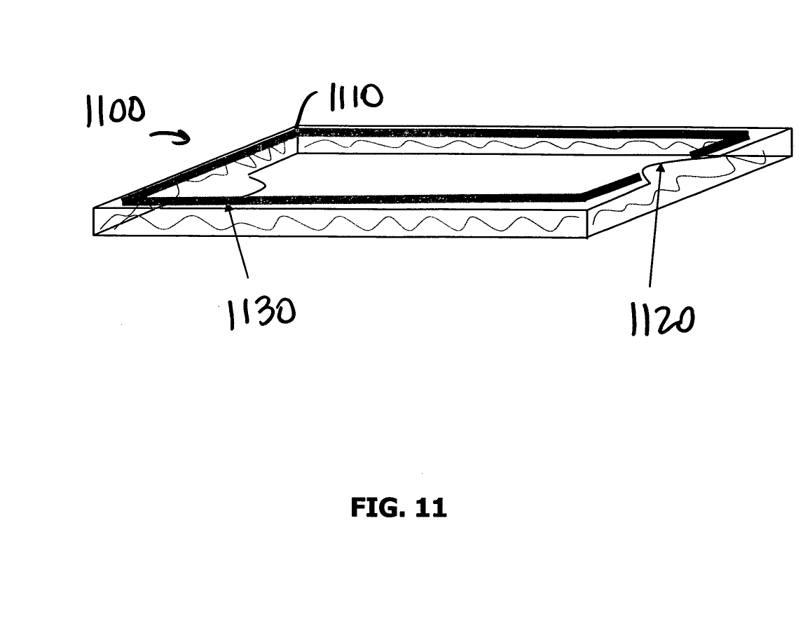

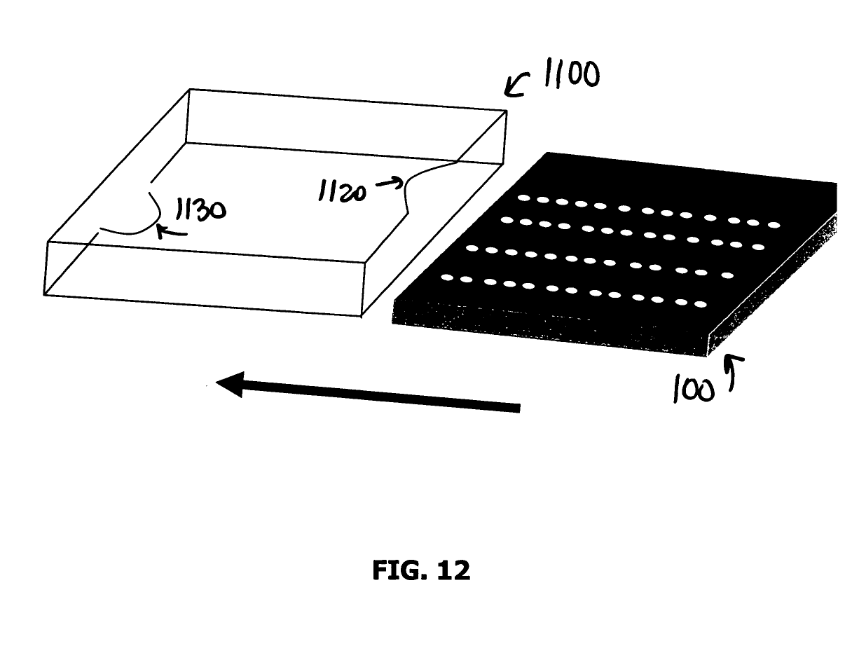

Referring now to FIG. 11, in accordance with another alternative preferred embodiment of the present invention, a sleeve type lid 1100 is shown. The multiwell plate 100 (or alternate multiwell plate 500) can slide into the sleeve type lid 1100 (as shown in FIG. 12), thereby both upper and lower well arrays are covered with just one lid, rather than two. Also contemplated are gripper cut-out areas 1120 and 1130, located anywhere on lid 1100, but preferably (as shown) at the ends of the top and bottom portions of the sleeve type lid 1100, respectively, to provide easy access to the multiwell plate 100 housed inside the sleeve type lid 1100, as can be seen in FIG. 12. Alternate multiwell plate 500 can also use this sleeve type lid, though not shown in FIG. 12.

The sleeve type lid 1100 may be thermoformed or molded from a clear or opaque polymer. The polymer on the top and bottom portions of the sleeve type lid 1100 preferably has a gas permeable sheet to allow for gas exchange of the wells and may be supported by a rigid frame 1110 found along the perimeter and on sides of lid 1100.

The various multiwell plates and/or lids described herein may have identifiers or tags associated with them, such that a user can track which of the upper or lower well arrays has been previously utilized, seeded, etc. These identifiers may be of any type desired, but some examples are labels with numerals (e.g. No. 1 and No. 2) placed on each of the upper and lower sides of the plate, or visible etchings anywhere or on the sides of the multiwell plate.

It should be noted that all figures described supra are not of actual size but represent accurate renditions and structural block diagrams of the preferred embodiments of the present invention.

Several commercial applications are contemplated for use with the embodiments of the present invention such as, but not limited to, for instance, applications involving PAMPA and IAM as previously mentioned.

Having described various preferred embodiments of the present invention, it will be apparent to those skilled in the art that various modifications and variations can be made to the present invention without departing from the spirit and scope of the invention. Thus, it is intended that the present invention covers the modifications and variations of this invention provided they come within the scope of the appended claims and their equivalents.

1. A multiwell plate apparatus, said apparatus comprising:
a plurality of first wells forming a first array;
a plurality of second wells, forming a second array, aligned with said first array of first wells; and
bottom portions of said first and second arrays of wells coupled together and having a permeable membrane at their interface.

2. The multiwell plate apparatus of claim 1 wherein each well of said first array has a respective well opening at the top of each well, said respective well openings facing up and wherein each well of said second array has a respective well opening at the top of each well, said respective well openings facing down.

3. The multiwell plate apparatus of claim 2 wherein said well openings of said second array are accessible by inverting or flipping said multiwell plate upside-down, thereby positioning said second array such that said respective well openings at the top of each well are facing up and positioning the first array such that said respective well openings at the top of each well are facing down.

4. The multiwell plate apparatus of claim 3 wherein fluid in the wells of both said first and second arrays is retained within the wells due to surface tension or capillary forces.

5. The multiwell plate apparatus of claim 1 wherein said permeable membrane is bonded midway through said first and second arrays of wells.

6. The multiwell plate apparatus of claim 1 wherein the permeable membrane is preferably a track-etch membrane.

7. The multiwell plate apparatus of claim 1 further comprising at least one rigid lid for covering said first and second arrays of wells.

8. The multiwell plate apparatus of claim 7 wherein said lid is compatible for robotic handling.

9. The multiwell plate apparatus of claim 7 wherein said lid is gas permeable on at least a portion of said lid.

10. The multiwell plate apparatus of claim 9 wherein said gas permeable lid comprises a thin polymer sheet or discs that allow for gas exchange.

11. The multiwell plate apparatus of claim 10 wherein said gas permeable lid further comprises an elastomeric gasket that fits into a recess on said multiwell plate.

12. The multiwell plate apparatus of claim 7 further comprising a sleeve-type lid covering said first and second arrays of wells.

13. The multiwell plate apparatus of claim 12 wherein said sleeve-type lid is thermoformed or molded from a polymer.

14. The multiwell plate apparatus of claim 12 wherein said sleeve-type lid further comprises means for accessing said multiwell plate when covered by said lid.

15. The multiwell plate apparatus of claim 14 wherein said means for accessing said multiwell plate are gripper cut-out areas on top and bottom sides of said sleeve-type lid.

16. The multiwell plate apparatus of claim 1 wherein said wells of said first and second arrays are patterned on said permeable membrane to enable cell binding.

17. The multiwell plate apparatus of claim 16 wherein said patterning on said permeable membrane is accomplished by a hydrophilic interaction, specific molecular interaction, coating with a lipid solution, or lamination.

18. The multiwell plate apparatus of claim 17 wherein said permeable membrane further comprises non-well regions on said permeable membrane patterned around said first and second well arrays to prevent cell binding or coating with lipid solutions.

19. The multiwell plate apparatus of claim 18 wherein said patterning may be a coating or lamination with a hydrophobic material.

20. The multiwell plate apparatus of claim 19 wherein said patterned permeable membrane is substantially flat.
